(19) United States
(12) Patent Application Publication
(10) Pub. No.: US 2005/0282286 A1
(43) Pub. Date: Dec. 22, 2005

(54) Method for determining organically bound carbon (TOC)

(75) Inventors: Claudia Rieger, Duesseldorf (DE); Ulrike Schweden, Ratingen (DE); Markus Lenhard, Viersen (DE); Elmar Grabert, Herne (DE); Ulrich Lundgreen, Duesseldorf (DE); Aria Farjam, Vaals (NL)

Correspondence Address: COCHRAN FREUND & YOUNG LLC, 2026 CARIBOU DR, SUITE 200, FORT COLLINS, CO, 80525, US

(73) Assignee: Hach Lange GmbH, Berlin (DE)

(21) Appl. No.: 11/145,533

(22) Filed: Jun. 3, 2005

(30) Foreign Application Priority Data
Jun. 9, 2004 (DE) 10 2004 028 270.6
Aug. 9, 2004 (DE) 10 2004 038 607.2

(51) Int. Cl. G01N 33/00

(52) U.S. Cl. 436/146

(57) ABSTRACT

The present invention relates to a method for determining the organically bound carbon (TOC) in an apparatus which has at least one reaction zone (2) and at least one detection zone (3),
a) the sample being placed into the reaction zone (2) of the apparatus (1), b) the apparatus being sealed, c) the organically bound carbon being converted into gaseous carbon dioxide by means of physical, chemical, biochemical or microbiological methods,
d) the gaseous carbon dioxide being transferred to the detection vessel (3, 3a) and e) the carbon dioxide content being determined by means of measurement methods known per se on the basis of the colour change of the indicator, after step a), the inorganic carbon being converted into carbon dioxide and expelled.

The present invention relates to a method for determining the organically bound carbon (TOC).

DE 19616760 A1 discloses a method and an apparatus for the continuous determination of the TOC value. In the method, the sample solution is continuously pumped into a microreactor, heated in the microreactor to a predetermined temperature and the organic compound is continuously converted by oxidation in the sample solution under a defined elevated temperature and defined elevated pressure. The gaseous oxidation products from the sample are then drawn through a membrane by suction and passed into a measuring cell and measured there by a mass spectrometer.

WO 99/42824 A1 describes a method for determining the TOC content in liquids, in which method the sample solution is introduced into a reaction chamber for oxidation of the carbon and is transferred, by liquid entering the reaction chamber from outside, into a measuring cell connected to the reaction chamber. There, the carbon dioxide concentration is determined dynamically in the course of flow through the measuring cell, a base value of carbon dioxide concentration corresponding to the untreated liquid and also a maximum value of carbon dioxide concentration established by the carbon dioxide-enriched liquid in the course of flow through the measuring cell being measured, and then a difference between maximum value and base value being formed. The carbon dioxide concentration is determined on the basis of conductivity measurements.

For determination of the analytical parameter TOC (Total Organic Carbon) in water samples, test kits have existed for some years. For example, test kits have been developed as are described in EP 0 663 239 B1. Using the system described there, the analysis can be carried out rapidly and simply on site by personnel with little training and using inexpensive media. The test kit has two containers designed as cuvettes, that is to say one sample reception container and one analysis container, each of which have container openings at the top which can be closed by screw-on closure caps. The test kit also comprises an adaptor via which the container openings, after the closures have been removed, can be connected together gas tightly. The adaptor is provided with a semipermeable membrane which is permeable to gases and, here, in particular, the constituent to be analysed, and the carrier gas. For this, it can consist, for example, of hydrophobic material. The analysis container can comprise the indicator reagent in preformulated and storable form. Likewise, the sample reception container can also be preformulated with a digestion reagent which converts the constituent to be analysed into the gas form.

DE 10018784 C2 describes a modification of this test kit. Here, the analysis container has a pressure-relief device which is preferably disposed at the end of the analysis container opposite the container opening. Excess carrier gas escapes through the pressure relief device which is only permeable to gases, especially when a liquid is used as indicator reagent.

WO 00/75653 describes an analysis apparatus which consists of two vessels which can be inserted one inside the other. Here, the inner vessel comprises the indicator. The sample to be analysed is located in the outer vessel. Both vessels are connected to one another only via the gas space. By heating, the volatile substances are transported from the sample into the gas phase and, via the gas space, come into contact with the indicator and then produce a change. The change in the indicator is determined by means of transmission of a light beam.

DE 10121999 A1 describes a method for the photometric or fluorimetric determination of volatile substances in solutions. For this, a system is used which has a cuvette which is divided into two zones by an ion-impermeable, gas-permeable membrane. By this means, the cuvette has two separate spaces for sample and digestion solution on the one side and indicator solution on the other.

DE 2534620 A1 discloses a method for determining the inorganic carbon content of aqueous liquids, in which a sample of the liquid to be analysed is introduced together with a carbon-dioxide-free carrier gas into a heated reaction chamber, the inorganic carbon compounds are decomposed by a reaction medium to form carbon dioxide, and the resultant carbon dioxide is fed to a CO₂ analyser. Movement of the reaction chamber is not mentioned.

To determine the TOC, it is regularly necessary to remove the inorganic carbon. For instance, DE 19906151 A1, DE 19616760 A1, DE 4307814 A1, DE 10018784 C2, DE 10121999 A1, EP 0663239 B1 and WO 00/75653 disclose that the inorganic carbon compounds can be removed by acidification and subsequent expulsion.

The test kits described which have existed for some years consequently have the following typical operating sequence in the analytical procedure:
1. the inorganic carbon of the sample (IC, inorganic carbon), consisting of physically dissolved carbon dioxide gas and chemically bound carbonates, is converted into carbon dioxide after acidification, and expelled,
2. the sample depleted by the IC is placed into the reaction zone of the TOC test kit,
3. a chemical oxidizing agent is added,
4. the reaction zone is connected via the gas space to an indicator solution which comprises a colour reagent sensitive to carbon dioxide,
5. the reaction zone is heated, the chemically bound organic carbon being converted by the oxidizing agent into carbon dioxide, and this gas is passed over into the indicator solution,
6. the reaction zone is cooled,
7. the colour change of the indicator solution due to the carbon dioxide passed over is measured in a photometer as extinction and, from the extinction, by means of available calibration data, the TOC is calculated,
8. the reaction zones used (cuvettes) together with the reagents used are placed back into the test kit packaging and later sent back to the supplier for correct disposal.

The first step, the expulsion of the IC, is generally carried out in the previously known methods as follows:
1. the sample is placed into an expulsion container, for example a conical flask,
2. the sample is acidified by adding an acid,
3. a magnetic stirring bar is placed in the expulsion container having the acidified sample,
4. the sample is placed on a magnetic stirrer and stirred for 5 to 10 minutes (the IC is expelled during this),
5. by means of a pipette, an aliquot of the IC-depleted sample is placed into the reaction space of the cuvette test,
6. expulsion container and magnetic stirring bar are cleaned.

This described handling is very complex for the user. Apart from this, the necessary cleaning work on the expulsion container and magnetic stirrer harbour the risk of contamination and thus falsification of the later sample analyses.

DE 4307814 A1 discloses, for the removal of the inorganic carbon, setting the sample to be studied to a weak pH of about 2 by means of an acid, e.g. hydrochloric acid, and bubbling the measurement amount by blowing in a gas, e.g. air. As a result of the reaction between the carbonates and the acid, carbon dioxide is formed. The outgassing of the carbon dioxide is achieved by the means that the sample is brought to overflow through an upwards-directed measurement line open at the top and the exiting carbon dioxide including the amount of gas previously blown in is removed. This method is also complex. Apart from this, the risk of inaccuracy of analysis is associated with the fact that water is drained from the container.

It is accordingly an object of the present invention to provide a method for determining the organically bound carbon (TOC) in an apparatus,
a) the sample being placed into the reaction zone of the apparatus,
b) the inorganic carbon being expelled,
c) the apparatus being sealed,
d) the organically bound carbon being converted into gaseous carbon dioxide by means of physical, chemical, biochemical or microbiological methods,
e) the gaseous carbon dioxide being transferred to the detection zone and
f) the carbon dioxide content being determined by means of measurement methods known per se on the basis of the colour change of the indicator.

By this method the process of conversion and expulsion of the inorganically bound carbon from the sample is to be markedly simplified, and the risks of contamination eliminated. At the same time, test kits are to be provided in which, in the reaction zone, an acidic reagent and/or an oxidizing reagent is already preformulated (packaged ready for use by the manufacturer) and the working step of acidification and/or addition of oxidizing agent to the sample is eliminated. Moreover, only one reaction zone is to be employed, that is to say expulsion of the inorganic carbon and/or oxidation of the organically bound carbon is to be carried out in one and the same reaction container. This also avoids the transfer of the sample to the reaction cuvette. In addition, the use of a magnetic stirring bar is to be avoided. In summary, this means that an object of the present invention is to provide a minimal system in which the inorganic carbon is to be expelled in the reaction zone. By this means a plurality of analyses are to be expelled in parallel.

This object is achieved by means of the fact that in step b), to expel the carbon dioxide formed by conversion of the inorganic carbon, the reaction zone (2) is agitated.

The removal of the inorganic carbon is made possible by adding an acid to the sample.

As acids, use is preferably made of phosphoric acid and sulphuric acid, or buffers derived therefrom. Very particular preference is given to phosphoric acid or the buffers derived therefrom.

By agitating the sample in the reaction zone, processes can be accelerated which contribute to the carbon dioxide being released rapidly into the surroundings from the reaction zone having the acidified sample. These are essentially serial equilibrium processes. These include:
Mixing the liquid sample for continuous transport of new carbon dioxide from the depths of the solution to the liquid-gaseous interface.
Mixing the gas space in the reaction zone for continuous transport of new carbon dioxide from the depths of the gas space to the opening of the reaction zone.
Transporting away into the surrounding gas space the carbon dioxide exiting at the opening.

According to the invention, the reaction zone or the reaction vessel is preferably agitated horizontally. However, this does not exclude vertical agitations also being carried out. Particular preference is given to the agitations being horizontal and circular. Likewise, the agitations can also be vertical, horizontal and circular in combination, as is the case, for example, with a tumbling agitation. These agitations produce a shaking, jolting and swinging, tumbling and therefore a vigorous agitation of the reaction zone. Preference is given to agitations which accelerate the abovementioned mixing and transport processes and secondly prevent the sample from spraying out of the reaction zone. Agitations have proved advantageous here which have a centrifugal force component, so that the sample is pressed onto the walls of the reaction zone and, together with the reaction zone structuring described below (for example shoulder), quantitative retention in the reaction zone is ensured. On the inner wall of the reaction zone, chicanes and structures can be mounted, which additionally mix the sample passing above them.

According to the invention, preference is given to the radius of the orbit being between 0.1 and 100 mm, preferably 0.5 and 5 mm. Very particular preference is given to a radius of 2 mm.

The angular frequency is preferably between 0.1 and 1000 Hz, particularly preferably between 1 and 100 Hz. Very particular preference is given to 30 Hz.

In addition to the agitation of the reaction zone which can accelerate the abovedescribed mixing and transport processes, further measures can prevent the sample from being sprayed out of the vigorously agitated reaction vessel.

For this, in a variant preferred according to the invention, a reaction zone or reaction vessel can be used which, below its opening, has a curvature in the form of a shoulder. That is to say the cuvette does not have the same diameter from the bottom to the opening. Rather, the diameter of the vessel constricts towards the opening. It has been found according to the invention that the arrangement of this shoulder completely avoids the sample from being sprayed out as a result of the agitation.

Here, by altering the shaping of the shoulder (for example sharper indentation, additional retention rim), the retention effect can be increased, the mechanical agitation intensified and thus finally the expulsion time shortened. Furthermore, structures can be mounted within the shoulder region which, in a similar manner to a deflection plate, guide the exiting sample back into the reaction zone. Of course, it is also possible to provide the closure of the reaction zone with structures (for example a shoulder).

Owing to the design with shoulder, operation of the reaction zone in vertical or horizontal form is made possible. In particular, the diameter constriction in the region of the shoulder has the advantage that the reaction zone can be agitated horizontally without the reaction solution and/or the sample exiting. In this case, all abovedescribed agitations are possible. Preferably, agitation can be about the axis of rotation. In this case, a liquid film forms on the inner wall of the reaction zone, which causes an accelerated gas exchange. Furthermore, other agitations in horizontal and vertical direction are also possible, for example jolting and rocking.

In a particularly inventively preferred variant, the reaction zone is used in an apparatus which may receive one or more reaction zones. This apparatus carries out the described agitations. By using the described apparatus for a plurality of reaction zones, parallel preparation of a plurality of samples for analysis is made possible. In the conventional methods, the sample preparation was time-consuming, inter alia, also because some of the typical users regularly had only a single magnetic stirrer available. The simultaneous makeup and/or the simultaneous preparation of a plurality of samples for analysis was therefore impossible. That is to say, in the previously known method for sample preparation by means of magnetic stirrer and conical flask, a majority of samples could only be prepared in sequence and thus in a significantly more time-consuming manner.

The described inventive acidification of the sample for conversion of the inorganic carbon into carbon dioxide now makes a significantly simpler handling of samples possible. In particular, it is now possible that, in the reaction zone, an acidic solution already preformulated can be supplied in finished package form by the manufacturer. The working step of acidification in a separate vessel, for example in a conical flask, by a magnetic stirrer, is eliminated completely. The cleaning and flushing of the conical flask, which is labour-consuming and time-consuming is likewise eliminated. Advantageously, acidification and subsequent conversion of the inorganic carbon and also subsequent conversion of the organically bound carbon into carbon dioxide can be carried out in one and the same reaction zone. The typical problems which are associated with unsatisfactory cleaning of the vessels (that is to say of the conical flasks used in the acidification), are thus avoided according to the invention.

The invention thus also relates to a test kit for determining the organically bound carbon (TOC) which comprises at least one reaction vessel or one reaction zone, the reaction zone comprising substances for producing carbon dioxide from the sample in preformulated and storable form and the detection zone comprising at least one gas-sensitive reagent in solid or liquid, and also preformulated and storable, form. The reaction zone is distinguished in this case in that it comprises, in preformulated form, acids for converting the inorganic carbon into carbon dioxide.

Preference is given to an embodiment in which the reaction zone in addition also comprises substances in preformulated and storable form for the conversion (oxidizing agent) of the TOC present in the sample to the gaseous carbon dioxide for the step d) to be carried out later. In this case, special measures can be taken to prevent the oxidizing agents present during the expulsion of the inorganic carbon in step b) from already converting TOC into carbon dioxide:
adding solid oxidizing agents which do not dissolve in the cold or dissolve only slightly
reducing the surface area of the solid oxidizing agents by compacting (for example tableting)
using oxidizing agents which do not oxidize TOC in the cold.

According to the invention, it is not excluded, however, in this case that the preformulated and gas-sensitive reagents and also substances for the conversion into carbon dioxide are stored outside the reaction zone or reaction vessel or detection zone or detection vessel and are not placed into the zones or vessels until the actual case of use. This applies in particular to the substances which are introduced into the reaction zone or the reaction vessel. Here, according to the invention, after addition of the sample, first the acid for the conversion and expulsion of the inorganic carbon can be added. Not until after completion of this reaction is the conversion of the organically bound carbon into carbon dioxide then carried out.

In a further variant of the inventive method, an air stream is passed via the opening of the reaction zone or reaction vessel. By this means, to a certain extent a suction action is generated so that the expulsion of the carbon dioxide from the opening of the reaction zone or reaction vessel is accelerated. Preferably, prepurified ambient air is used for this.

The air can be agitated in the simplest case by disposition of a fan. It is likewise possible, however, to pass a targeted air current over the opening of the reaction zone or reaction vessel by means of a nozzle. Owing to the associated higher velocity of the air stream, a further acceleration of the transport of the carbon dioxide gas from the reaction zone or reaction vessel can be achieved.

Likewise, however, it is also possible to accelerate the gas discharge via a pulsed air stream. Such a pulsation can be achieved, in the simplest case, by a continuous deflection of the air stream being performed in consequence of the agitations of the reaction zone or reaction vessel. In this case, by means of the pulsation, likewise, the sample situated in the reaction zone can be agitated in a pulsed manner, an additional mixing in the liquid sample being performed, which in turn leads to an accelerated expulsion.

The invention will be described in more detail below with reference to the drawings:

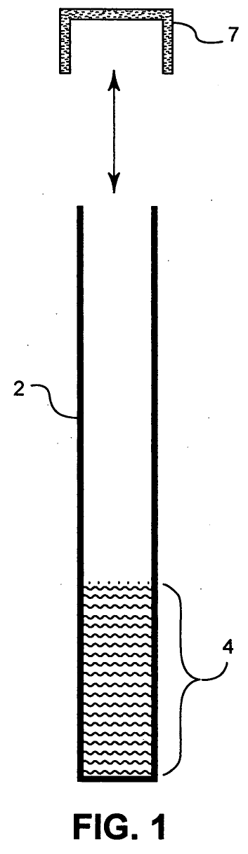

FIG. 1 shows an example of a reaction zone which can be used according to the invention. This zone essentially consists of a closable container having a closing apparatus 7, preferably a screw closure cap. After removal of the cap 7, the sample is placed into the reaction zone 2. This reaction zone preferably comprises according to the invention a preformulated reaction solution 4. This reaction solution can comprise, firstly, reagents for converting the inorganically bound carbon and, secondly, also reagents for converting the organically bound carbon. It is likewise also possible, however, firstly to place the sample into the reaction zone 2 and then to use a conversion solution for the inorganic carbon, which solution is co-supplied by the manufacturer. This is generally acids. After addition of the acids and of the sample, the carbon dioxide being released is expelled according to the invention by means of agitation. Further handling or procedure of the analysis can be performed in one of the apparatuses 2 to 5. The inventive use, however, is not limited to these apparatuses.

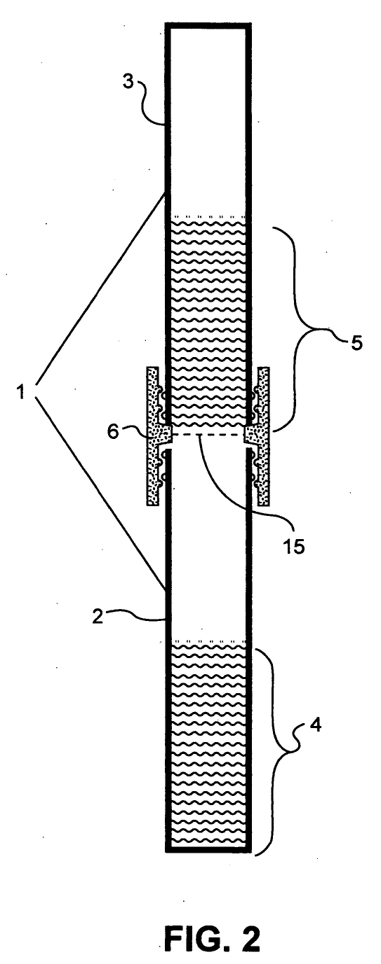

FIG. 2 shows an example of an apparatus as disclosed by EP 0 663 239 A2. Here, in a similar manner to the description for FIG. 1, after addition of the acids and the sample to the reaction zone 2, the carbon dioxide being released is expelled according to the invention by means of agitation. Thereafter, the reaction zone 2 is connected to the detection zone 3. In the reaction zone 2, the reaction solution 4 is situated, and in the detection zone 3, the indicator solution 5 is situated. The two zones are connected via the adaptor 6 which has a membrane 15. For generating the gaseous constituents from the sample 4, physical, chemical, biochemical or microbiological methods can be employed. Chemical methods which may be mentioned are preferably acidification, alkalization, oxidation, reduction and derivatization. Methods for accelerating the gas formation are described, for example, in EP 0 663 239 B1. Preferably, a treatment with oxidizing agent, for example persulphate, is performed, and the reaction zone 2 is heated. The chemically bound organic carbon is converted into carbon dioxide by the oxidizing agent and this gas is passed over to the indicator solution 5. The reaction zone is cooled. The colour change of the indicator solution due to the carbon dioxide which is passed over is measured as extinction in a photometer. The TOC is calculated from the extinction by means of available calibration data.

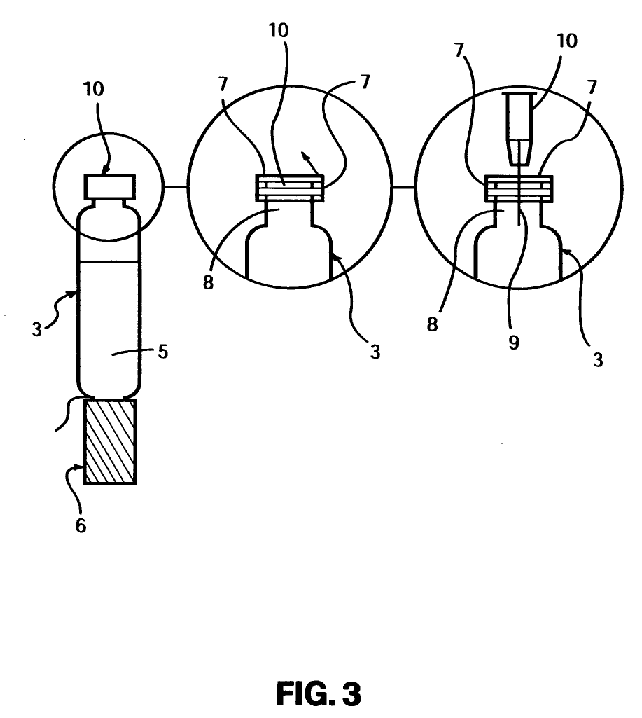

FIG. 3 shows a test kit as is disclosed by DE 10018784 C2. In contrast to the test kit according to FIG. 2, here, a connection of the detection zone 3 to the outside atmosphere is provided. The gas forced via the adaptor 6 into the indicator solution 5 of the reaction vessel 2 is measured as described in the example according to FIG. 2. By means of the needle 10, the closure 7 seated on the opening 8 can be opened towards the outside, in order to produce in this manner a pressure relief via the cannula 9. This can contribute to an additional acceleration of the gas transport. Here, in a similar manner to the description to FIG. 1, after addition of the acids and the sample to the reaction zone, which is developed in a similar manner to FIG. 2, the carbon dioxide being released is expelled according to the invention by means of agitation.

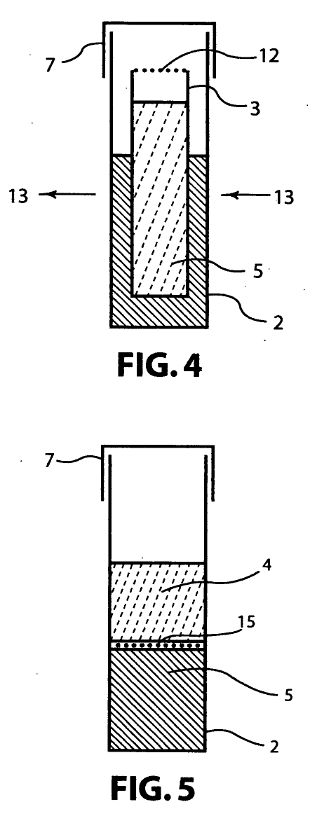

FIG. 4 further shows an apparatus as disclosed by WO 00175653. In this example, the detection zone 3 having the indicator solution 5 is inserted into the reaction zone 2. The vessels are connected to one another via the gas space. Here, in a similar manner to the description to FIG. 1, after addition of the acids and the sample to the reaction zone 2, the carbon dioxide being released is expelled according to the invention by means of agitation. By heating, carbon dioxide produced from the bound organic carbon is transported from the sample and brought into contact with the indicator 5. The change in the indicator is measured by means of the transmission of a light beam 13.

In a particular embodiment, reaction zone and detection zone can be connected to one another via a membrane 12.

FIG. 5 further shows an example of a test kit as disclosed by DE 10121999 A1. The indicator solution 5 is separated from the reaction solution 4 by a membrane 15. Over the reaction solution 4 are situated a gas space 14 and the closure cap 7. For determination of the TOC, the cuvette is opened by its closure lid 7 and, in a similar manner to the description to FIG. 1, after addition of the acids and the sample to the reaction zone 2, the carbon dioxide being released is expelled according to the invention by means of agitation. After it is closed by the lid 7, the cuvette is turned round and placed in a thermostat where, by heating the reaction zone 4, carbon dioxide is produced from the bound organic carbon and is transported from the sample and brought into contact with the indicator 5. Subsequently thereto, the colour change of the indicator 5 is measured.

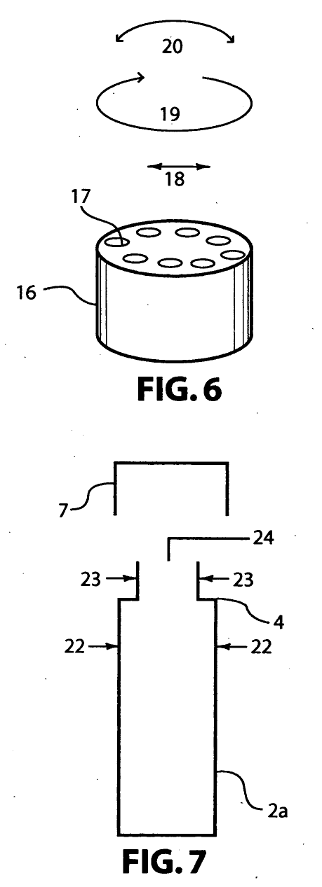

FIG. 6 shows an inventively preferred variant for use in the inventive method. In addition to acidification of the sample to remove the inorganic carbon, as an additional measure, agitation of the reaction zone 2 is provided. These agitations can be performed in the form of circular motions or horizontal motion to and fro. In the variant according to FIG. 6, for this purpose, the reaction zone can be inserted into the apparatus 16. In this apparatus, a plurality of openings 17 are preferably provided. By this means, a multiplicity of reaction zones can be inserted simultaneously into the apparatus. Agitation in the horizontal 18 mixes the reaction solution with the conversion agent, for which acids are preferably used. In the example according to FIG. 7, a preferred circular motion 19 is also provided. Tilting agitations 20 or tumbling agitations are likewise possible, and also a combination of all types of agitation.

FIG. 7 shows a preferred embodiment of the reaction zone 2a. This zone likewise has a closure apparatus 7. In contrast to the apparatus according to FIG. 1, the reaction zone 2a is equipped with a shoulder 21. This constricts the diameter 22 to the diameter 23 of the opening 24. When this cuvette is employed, this cuvette, in combination with the apparatus according to FIG. 6, achieves a particularly optimum agitation frequency. Reaction solution and acidulant are mixed in such a manner as to achieve an acceleration of the exit of the carbon dioxide formed from the inorganic carbon and of the dissolved carbon dioxide present in the sample, at the same time, exit from the reaction zone is avoided.

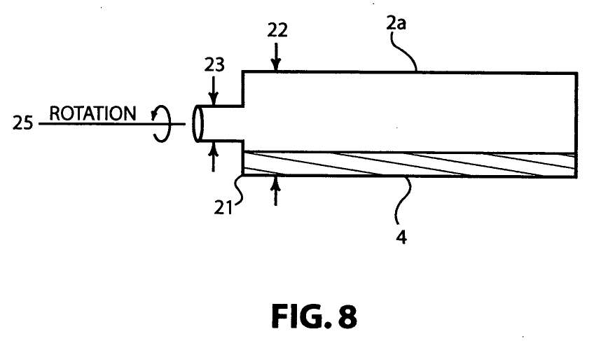

FIG. 8 shows a preferred embodiment of the reaction zone 2. The reaction zone is likewise equipped with a shoulder 21. By means of the diameter constriction, the reaction zone can be agitated in a lying position, without the reaction solution and/or sample 4 exiting. In this case, all abovedescribed agitations are possible. Preference is given to an agitation about the axis of rotation 25. In this case a liquid film forms on the inner wall of the reaction zone, which liquid film causes an accelerated gas exchange.

1. Method for determining the organically bound carbon (TOC) in an apparatus which comprises at least one reaction zone (2) and at least one detection zone (3),
a) the sample being placed into the reaction zone (2) of the apparatus (1),
b) the inorganic carbon being expelled,
c) the apparatus being sealed,
d) the organically bound carbon being converted into gaseous carbon dioxide by means of physical, chemical, biochemical or microbiological methods,
e) the gaseous carbon dioxide being transferred to the detection zone (3, 3a) and
f) the carbon dioxide content being determined by means of measurement methods known per se on the basis of the colour change of the indicator,
characterized in that, in step b), to expel the carbon dioxide formed by conversion of the inorganic carbon, the reaction zone (2) is agitated.

2. Method according to claim 1, characterized in that, in step b), the inorganic carbon is converted into carbon dioxide by adding acid(s).

3. Method according to claim 2, characterized in that, as acid(s) for converting the inorganic carbon, use is made in reaction zone (2) of phosphoric acid, sulphuric acid and buffers derived therefrom.

4. Method according to claim 3, characterized in that the acids in reaction zone (2) are present in ready-to-use formulation.

5. Method according to one of claims 1 to 4, characterized in that the reaction zone (2) is agitated horizontally.

6. Method according to claim 5, characterized in that the reaction zone (2) is agitated horizontally and circularly.

7. Method according to claim 6, characterized in that the radius of the orbit of the reaction zone (2) is between 0.1 and 100 mm.

8. Method according to one of claims 6 or 7, characterized in that the angular frequency of the reaction zone (2) is between 0.1 and 1000 Hz.

9. Method according to one of the preceding claims, characterized in that a reaction zone (2) is used which has a curvature (shoulder) below the opening.

10. Method according to claim 9, characterized in that the reaction zone (2) is used vertically or horizontally.

11. Method according to claim 10, characterized in that the horizontal reaction zone (2) is rotated about its horizontally directed axis.

12. Method according to one of the preceding claims, characterized in that, above the opening of the reaction zone (2), an air stream is generated which produces an exchange of the gas space within the reaction zone with the ambient air.

13. Method according to claim 12, characterized in that the ambient air is prepurified.

14. Method according to one of claims 12 or 13, characterized in that the air is agitated by means of a fan.

15. Method according to one of claims 12 to 14, characterized in that the air stream is directed in the form of a laminar flow against the opening of the reaction zone (2).

16. Method according to one of claims 12 to 15, characterized in that a pulsed air stream is produced.

17. Test kit for carrying out a method according to one of the preceding claims, comprising at least one reaction zone (2) and at least one detection zone (3) in preformulated and storable form,
a) at least one agent for converting the inorganic carbon into carbon dioxide,
b) at least one agent for converting the bound organic carbon for the conversion into carbon dioxide, preferably an oxidizing agent, and
c) at least one gas-sensitive reagent which experiences a colour change on contact with carbon dioxide,
and also an apparatus (16) for agitating the reaction zone.

18. Test kit according to claim 17, characterized in that the agent a) is present in the reaction zone in preformulated and storable form.

19. Test kit according to claim 18, characterized in that agent b) is present in the reaction zone in preformulated and storable form.

20. Test kit according to claim 19, characterized in that the agents a) and b) are present in the reaction zone in preformulated and storable form.

21. Use of the test kit according to one of claims 17 to 20 for determining the organically bound carbon (TOC).
